(19) United States
(12) Patent Application Publication
(10) Pub. No.: US 2017/0345633 A1
(43) Pub. Date: Nov. 30, 2017

(54) IMAGE ACQUISITION SEMICONDUCTOR FILM FOR HIGH-RESOLUTION MASS SPECTROMETRIC IMAGING SYSTEM, PREPARATION METHOD, AND APPLICATION

(71) Applicant: CENTRAL CHINA NORMAL UNIVERSITY, HUBEI (CN)

(72) Inventors: Hongying ZHONG, Hubei (CN); Lulu HUANG, Hubei (CN); Xuemei TANG, Hubei (CN); Wenyang ZHANG, Hubei (CN)

(73) Assignee: CENTRAL CHINA NORMAL UNIVERSITY, HUBEI (CN)

(21) Appl. No.: 15/529,515

(22) PCT Filed: Jan. 15, 2016
(86) PCT No.: PCT/CN2016/071039

§ 371 Date: May 25, 2017

(30) Foreign Application Priority Data
Jan. 21, 2015 (CN) 201510030221.8

(51) Int. Cl. H01J 49/04; G02B 26/10; G01N 33/483; H01J 49/00; G01N 1/28

(52) U.S. Cl. CPC H01J 49/0418; H01J 49/0031; G01N 1/2813; G01N 33/4833; G02B 26/10

(57) ABSTRACT

An image acquisition semiconductor film for a high-resolution mass spectrometric imaging system, and a preparation method and an application. The image acquisition semiconductor film for the high-resolution mass spectrometric imaging system is prepared by using the following method: weighing semiconductor nanometer particles, putting the semiconductor nanometer particles into a muffle furnace for burning first, further grinding by using an agate mortar, and uniformly dispersing the semiconductor nanometer particles so as to obtain semiconductor nanometer powder; and finally, pressing the semiconductor nanometer powder in a compressor so as to obtain the semiconductor film. Based on laser activated electron tunnelling as well as photoelectron capture ionization and dissociation, sample molecules are ionized without background interference; the limitation of a conventional MALDI substrate is overcome; the semiconductor film is simple and easy to obtain, is stable in mass spectrometric signal, has a uniform and smooth surface, generates no background interference, and can be used for fingerprint analyzing and animal and plant tissue slice analysis; and the semiconductor film is particularly suitable for accurate mass spectrometric imaging of small molecular substances, so that quality control and industrialization can be performed conveniently.

### BACKGROUND OF THE INVENTION

### 1. Field of the Invention

The present invention relates to the field of mass spectrometric imaging, in particular, to an image acquisition semiconductor film for a high-resolution mass spectrometric imaging system, a preparation method, and an application.

### 2. Description of Related Art

The matrix-assisted laser desorption/ionization mass spectrometry is a common analysis technology in current mass spectrometric imaging. In this technology, an organic small-molecule matrix capable of absorbing laser energy is covered on a surface of a tissue slice and transfers the energy to sample molecules to vaporize and ionize them, which are further detected by a mass analyzer. In this technology, the co-crystallization of the organic small-molecule matrix and the sample molecules is the key because the resultant crystals directly affect the mass accuracy and resolution of an analysis result, reproducibility of an experimental result, and a quantitative capability.

In the prior art, a matrix is usually first dissolved by using an organic solvent, then, a matrix solution is sprayed on a surface of a tissue slice, and after the solvent volatilizes, a sample and molecules of the matrix form co-crystals. A major disadvantage of the prior art is difficulty in forming crystals that are uniform in size and that have controllable appearance. Therefore, spectra obtained by shotting the laser beam at different locations do not have reproducibility, and there is no quantitative relationship between signal intensity and a sample quantity. In addition, because of differences in crystal size and appearance, after sample molecules are bombarded by the laser, resultant ions have different initial speeds and directions. Therefore, spatial resolution of an image and mass accuracy are affected. Furthermore, the organic small-molecule matrix usually also generates a series of background peaks in the low-mass area, suppresses signals of low mass molecules, and severely contaminates the ion source.

### SUMMARY OF THE INVENTION

For disadvantages in the prior art, the present invention is directed to providing an image acquisition semiconductor film for a high-resolution mass spectrometric imaging system, a preparation method, and an application.

An image acquisition semiconductor film for a high-resolution mass spectrometric imaging system is obtained by, after burning semiconductor nanometer particles to remove organic impurities adsorbed on surfaces, grinding the semiconductor nanometer particles, and then placing the semiconductor nanometer particles into a compressor and subjected to a high pressure to make them into a film.

According to the foregoing solution, the semiconductor nanometer particles are (Bi₂O₃)_{0.07}(CoO)_{0.03}(ZnO)_{0.9} semiconductor nanometer particles.

According to the foregoing solution, a temperature of the burning is 350° C., and a time of the burning is 1 hour.

A preparation method of the foregoing image acquisition semiconductor film for a high-resolution mass spectrometric imaging system includes the following steps:

1) burning the semiconductor nanometer particles in a muffle furnace at 350° C. for 1 hour;

2) further levigating the semiconductor nanometer particles obtained in step 1) by using an agate mortar to uniformly disperse the semiconductor nanometer particles, so as to obtain semiconductor nanometer powder;

3) placing the semiconductor nanometer powder obtained in step 2) into a grinding tool of the compressor, then placing the nanoparticles into the compressor, and applying pressure to press the semiconductor nanometer powder to obtain a semiconductor film; and

4) taking out the semiconductor film obtained by pressing in step 3) and keeping the semiconductor film at a room temperature. According to the foregoing solution, the pressing in step 3) is pressing for 1 minute under the pressure of 2000 kg to 4800 kg.

The foregoing image acquisition semiconductor film for a high-resolution mass spectrometric imaging system is applied to latent fingerprint image analysis, animal tissue slice image analysis, or plant tissue slice image analysis.

According to the foregoing solution, the application is: after fixing or pressing a plant tissue slice, an animal tissue slice, or an latent fingerprint onto the image acquisition semiconductor film for a high-resolution mass spectrometric imaging system, fixing the semiconductor film onto a sample target, and directly placing the sample target into a mass spectrometer for analysis.

According to the foregoing solution, the application to latent fingerprint image analysis is: after directly pressing the fingerprint onto a surface of the semiconductor film, fixing the semiconductor film to a MALDI sample target, and placing the MALDI sample target into a mass spectrometer to perform laser desorption/ionization for image analysis.

According to the foregoing solution, the application to animal tissue slice image analysis is: first freezing a tissue slice at a temperature of −80° C., further slicing the animal tissue slice into a slice with thickness of 20 microns, directly transferring the slice onto a surface of the semiconductor film, fixing the semiconductor film onto a MALDI sample target, and after placing the MALDI sample target into a mass spectrometer, performing laser desorption/ionization for image analysis.

According to the foregoing solution, the application to plant tissue slice image analysis is: using the semiconductor film as a preliminary film, placing the plant tissue slice onto a surface of the preliminary film, further applying pressure, after filling the plant tissue slice into the nanometer particles of the semiconductor film, obtaining a semiconductor film including the plant tissue slice, fixing the semiconductor film onto a MALDI sample target, and after placing the MALDI sample target into a mass spectrometer, performing laser desorption/ionization for image analysis. In the present invention, a type and a dosage of semiconductor particles may be determined according to different samples. Semiconductor nanometer particles that have been burnt in the muffle furnace need to be levigated in an agate mortar to be uniformly dispersed, so that semiconductor films obtained by pressing have a uniform size and uniform thickness.

In the preparation method of the present invention, a film that is uniform and that has a controllable size and controllable thickness is prepared by pressing a semiconductor nanometer particle material under high pressure, so that indeterminacy in recrystallization by using an organic solvent in the prior art is avoided. The obtained semiconductor film can absorb ultraviolet light. Under irradiation of laser, electrons located in a valence band are excited to a conduction band and tunnel. The tunneling electrons are captured by a tissue slice or neutral molecules in a fingerprint, so as to trigger ionization and chemical bond breaking of sample molecules. Hence, imaging is further performed according to mass spectrometric signals. In addition, the image obtained by using the semiconductor film of the present invention has a stable signal, no background interference, a good linear relationship between signal intensity and a sample quantity, good reproductively, high sensitivity, and high spatial resolution.

The present invention has the following beneficial effects:

(1) As compared with an existing MALDI mass spectrometric imaging system, a current MALDI imaging technology does not have an image acquisition film. Usually, after being dissolved in an organic solvent, an organic small-molecule matrix covers a tissue slice in a spraying manner. Because co-crystal particles of the organic matrix and sample molecules have different sizes, a mass spectrum would be prone to have an unstable signal, a poor quantitative relationship, low resolution, and a great amount of background interference generated in a low-mass area; whereas in the present invention, a principle of a semiconductor nanometer material for capturing laser-induced tunneling electrons is used to ionize sample molecules, prevents background interference, and overcome limitation of a common MALDI matrix.

(2) The acquisition semiconductor film for a high-resolution mass spectrometric imaging system image according to the present invention can be obtained by pressing and modeling semiconductor nanometer particles under high pressure. Not only a method is simple, but also the obtained film is uniform. The obtained film has a controllable size and controllable thickness, stable properties, stable mass spectrometric signals, and a uniform and smooth surface, does not generate background interference, can be used in fingerprint analysis and animal/plant tissue slice analysis, is particularly suitable for accurate mass spectrometric imaging of a small-molecule substance, and is convenient for quality control and industrialization.

### BRIEF DESCRIPTION OF THE DRAWINGS

FIG. 1 is a mass spectrometric image obtained in Embodiment 1, in which imaging is constructed by using a dienestrol molecular ion peak, a fingerprint is pressed onto an image acquisition semiconductor film, and after the film is scanned by laser, the mass spectrometric image is obtained.

FIG. 2 is a mass spectrometric image of an Arabidopsis thaliana leave obtained in Embodiment 2, in which imaging is constructed by using a jasmonic acid molecular ion peak.

FIG. 3 is a mass spectrometric image of a mouse brain obtained in Embodiment 3, in which imaging is constructed by using a cephalin molecular ion peak.

### DESCRIPTION OF THE EMBODIMENTS

In order to provide a better understanding of the present invention, the embodiments will be described in the following to further elaborate the content of the present invention, but the content of the present invention is not limited by the following embodiments.

### Embodiment 1

In preparation of an image acquisition semiconductor film for high-resolution mass spectrometric imaging system, the film is applied to imaging analysis on an latent fingerprint, and operation steps are performed in sequence as follows:

1) weighing a particular amount, for example, 10 mg, of (Bi₂O₃)_{0.07}(CoO)_{0.03}(ZnO)_{0.9} semiconductor nanometer particles by using an analytic balance, where a type and a dosage of a material may be determined according to different samples;

2) burning the semiconductor nanometer particles obtained in step 1) in a muffle furnace at a temperature of 350° C. for 1 hour to remove contamination of attached organic molecules;

3) further levigating the semiconductor nanometer particles obtained in step 2) by using an agate mortar to uniformly disperse the semiconductor nanometer particles;

4) placing semiconductor nanometer powder obtained in step 3) into a compressor, then placing nanoparticles into the compressor, applying pressure of 4800 kg, and maintaining it for 1 minute under such pressure;

5) taking out the semiconductor film obtained by pressing in step 4) and keeping the semiconductor film at a room temperature;

6) pressing a fingerprint onto a surface of the semiconductor film obtained in step 5), fixing the film onto a surface of a MALDI sample target, and placing the MALDI sample target into a mass spectrometer to perform laser desorption/ionization for image analysis.

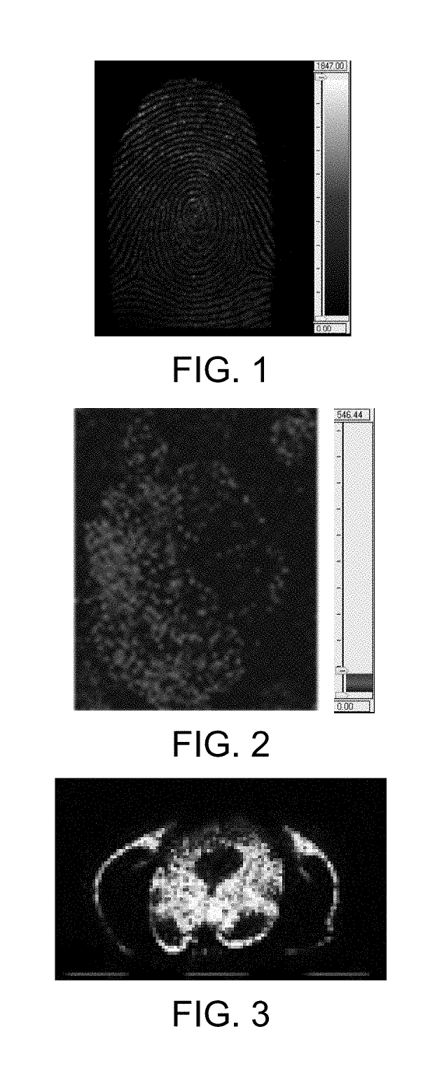

The mass spectrometric image obtained in this embodiment is shown in FIG. 1, and this image is a mass spectrometric image of oestrogen dienestrol. It can be seen from FIG. 1 that the image has a stable signal, no background interference, high sensitivity, and high resolution.

### Embodiment 2

In preparation of an image acquisition semiconductor film for high-resolution mass spectrometric imaging system, the film is applied to mass spectrometricimaging of phytohormone jasmonic acid, and operation steps are as follows:

1) weighing a particular amount, for example, 10 mg, of (Bi₂O₃)_{0.07}(CoO)_{0.03}(ZnO)_{0.9} semiconductor nanometer particles by using an analytic balance, where a type and a dosage of a material may be determined according to different samples;

2) burning the semiconductor nanometer particles obtained in step 1) in a muffle furnace at a temperature of 350° C. for 1 hour to remove contamination of attached organic molecules;

3) further levigating the semiconductor nanometer particles obtained in step 2) by using an agate mortar to uniformly disperse the semiconductor nanometer particles;

4) placing semiconductor nanometer powder obtained in step 3) into a compressor, then placing the nanoparticles into the compressor, applying pressure of 2000 kg, and maintaining it for 1 minute under such pressure to obtain a semiconductor film;

5) using the semiconductor film obtained by pressing in step 4) as a preliminary film, placing an Arabiclopsis thaliana leave onto a surface of the preliminary film, further placing it into a sheet press and increasing pressure to 2000 kg, maintaining it for 1 minute under such pressure to obtain a semiconductor film including the leave; and

6) fixing the semiconductor film obtained in step 5) onto a surface of a MALDI sample target, and placing the MALDI sample target into a mass spectrometer to perform laser desorption/ionization for imaging analysis.

The mass spectrometric image obtained in this embodiment is shown in FIG. 2, and this image is a mass spectrometric image of phytohormone jasmonic acid. It can be seen from FIG. 2 that the image has a stable signal, no background interference, high sensitivity, and high resolution.

### Embodiment 3

In preparation of an image acquisition semiconductor film for high-resolution mass spectrometric imaging system, the film is applied to mass spectrometric imaging of cephalin of a brain tissue, and operation steps are as follows:

1) weighing a particular amount, for example, 10 mg, of (Bi₂O₃)_{0.07}(CoO)_{0.03}(ZnO)_{0.9} semiconductor nanometer particles by using an analytic balance, where a type and a dosage of a material may be determined according to different samples;

2) burning the semiconductor nanometer particles obtained in step 1) in a muffle furnace at a temperature of 350° C. for 1 hour to remove contamination of attached organic molecules;

3) further levigating the semiconductor nanometer particles obtained in step 2) by using an agate mortar to uniformly disperse the semiconductor nanometer particles;

4) placing two thirds of semiconductor nanometer powder obtained in step 3) into a compressor, then placing nanoparticles into the compressor, applying pressure of 4,800 kg, and maintaining it for 1 minute under such pressure to obtain a semiconductor film;

5) taking out the semiconductor film obtained by pressing in step 4), after freezing a mouse brain at a temperature of −80° C., successively slicing the mouse brain, where the thickness of each slice is 20 microns, and directly transferring the slices in sequence onto a surface of the film; and

6) fixing the film obtained in step 5) onto a surface of a MALDI sample target, and placing the MALDI sample target into a mass spectrometer to perform laser desorption/ionization for imaging analysis.

The mass spectrometric image obtained in this embodiment is shown in FIG. 3, and this image is a mass spectrometric image of cephalin. It can be seen from FIG. 3 that the spectral image has a stable signal, no background interference, high sensitivity, and high resolution.

Apparently, the aforementioned embodiments are merely used as examples for describing the present invention more clearly, and are not used to limit the method for implementation. To those having ordinary skill in the art, various modifications and variations can be made based on the above description. All possible implementations could not and need not be exhaustively listed here. Therefore, all the obvious modifications and variations derived from here still fall within the protective scope of the present invention.

1. An image acquisition semiconductor film for a high-resolution mass spectrometric imaging system, wherein the image acquisition semiconductor film is obtained by, after burning semiconductor nanometer particles to remove organic impurities attached to surfaces, grinding the semiconductor nanometer particles, and then placing the semiconductor nanometer particles into a compressor to press them into a film, wherein the semiconductor nanometer particles are (Bi₂O₃)_{0.07}(CoO)_{0.03}(ZnO)_{0.9}, semiconductor nanometer particles.

2. The image acquisition semiconductor film for a high-resolution mass spectrometric imaging system according to claim 1, wherein a temperature of the burning is 350° C., and a time of the burning is 1 hour.

3. A preparation method of the image acquisition semiconductor film for a high-resolution mass spectrometric imaging system according to claim 1, comprising the following steps:
1) burning the semiconductor nanometer particles in a muffle furnace at 350° C. for 1 hour;
2) further levigating the semiconductor nanometer particles obtained in step 1) by using an agate mortar to uniformly disperse the semiconductor nanometer particles, so as to obtain semiconductor nanometer powder;
3) placing the semiconductor nanometer powder obtained in step 2) into a compressor, then placing nanoparticles into the compressor, and applying pressure to press the semiconductor nanometer powder to obtain a semiconductor film; and
4) taking out the semiconductor film obtained by pressing in step 3) and keeping the semiconductor film at a room temperature.

4. The preparation method according to claim 3, wherein the pressing in step 3) is pressing for 1 minute under the pressure of 2000 kg to 4800 kg.

5. An application of the image acquisition semiconductor film for a high-resolution mass spectrometric imaging system according to claim 1 to latent fingerprint image analysis, animal tissue slice image analysis, or plant tissue slice image analysis.

6. The application according to claim 5, wherein the application is: after fixing or pressing a plant tissue slice, an animal tissue slice, or an latent fingerprint onto the image acquisition semiconductor film for a high-resolution mass spectrometric imaging system, fixing the semiconductor film onto a sample target, and directly placing the sample target into a mass spectrometer for image analysis.

7. The application according to claim 5, wherein the application to latent fingerprint image analysis is: after directly pressing the latent fingerprint onto a surface of the semiconductor film, fixing the semiconductor film to a MALDI sample target, and placing the MALDI sample target into a mass spectrometer to perform laser desorption/ionization for image analysis.

8. The application according to claim 5, wherein the application to animal tissue slice image analysis is: first freezing an animal tissue slice at a temperature of −80° C., further slicing the animal tissue slice into a slice with thickness of 20 microns, directly transferring the slice onto a surface of the semiconductor film, fixing the semiconductor film onto a MALDI sample target, and after placing the MALDI sample target into a mass spectrometer, performing laser desorption/ionization for image analysis.

9. The application according to claim 5, wherein the application to plant tissue slice image analysis is: using the semiconductor film as a preliminary film, placing the plant tissue slice onto a surface of the preliminary film, further applying pressure, after filling the tissue slice into the nanometer particles of the semiconductor film, obtaining a semiconductor film comprising the plant tissue slice, then fixing the semiconductor film onto a MALDI sample target, and after placing the MALDI sample target into a mass spectrometer, performing laser desorption/ionization for image analysis.

10. The application according to claim 6, wherein the application to latent fingerprint image analysis is: after directly pressing the latent fingerprint onto a surface of the semiconductor film, fixing the semiconductor film to a MALDI sample target, and placing the MALDI sample target into the mass spectrometer to perform laser desorption/ionization for image analysis.

11. The application according to claim 6, wherein the application to animal tissue slice image analysis is: first freezing an animal tissue slice at a temperature of −80° C., further slicing the animal tissue slice into a slice with thickness of 20 microns, directly transferring the slice onto a surface of the semiconductor film, fixing the semiconductor film onto a MALDI sample target, and after placing the MALDI sample target into the mass spectrometer, performing laser desorption/ionization for image analysis.

12. The application according to claim 6, wherein the application to plant tissue slice image analysis is: using the semiconductor film as a preliminary film, placing the plant tissue slice onto a surface of the preliminary film, further applying pressure, after filling the tissue slice into the nanometer particles of the semiconductor film, obtaining a semiconductor film comprising the plant tissue slice, then fixing the semiconductor film onto a MALDI sample target, and after placing the MALDI sample target into the mass spectrometer, performing laser desorption/ionization for image analysis.
